(19) United States
(12) Patent Application Publication
(10) Pub. No.: US 2019/0388267 A1
(43) Pub. Date: Dec. 26, 2019

(54) Device to Alleviate Back Pain

(71) Applicant: Sunitha Bhaskaran, Los Gatos, CA (US)

(72) Inventor: Sunitha Bhaskaran, Los Gatos, CA (US)

(21) Appl. No.: 16/450,937

(22) Filed: Jun. 24, 2019

Related U.S. Application Data
(60) Provisional application No. 62/689,217, filed on Jun. 24, 2018.

(51) Int. Cl. A61F 7/02; A47C 7/42; A47C 7/46

(52) U.S. Cl. CPC A61F 7/02; A61F 2007/0024; A47C 7/46; A47C 7/425

(57) ABSTRACT

An article of back-support comprising of a flexible pad that can be customized to provide pain relief is disclosed. In preferred embodiments, the flexible pad comprises at least one material that substantially maintain its temperature for a substantial period of time to provide heat or cold therapy. In further preferred embodiments, the back-support mechanism can be rolled up or folded to provide targeted pain relief. Its thickness can be increased or decreased to suit the comforts of the user. The back-support mechanism is portable and can be easily attached to a garment or chair with fastening means.

### CROSS REFERENCE TO RELATED APPLICATIONS

This application claims the benefit of U.S. Provisional Application No. 62/689,217, filed Jun. 24, 2018, which is incorporated herein.

### FEDERALLY SPONSORED RESEARCH

Not Applicable

### SEQUENCE LISTING

Not Applicable

### FIELD OF THE INVENTION

The present patent specification relates to a novel back-support mechanism. More particularly, this patent specification relates to an apparatus that provides adjustable support to the back region.

### BACKGROUND

Eight out of ten Americans experience a painful back episode at some time during their lives and back pain is the single leading cause of disability worldwide.

Individuals who suffer from back pain, struggle to be seated upright on a flat surface without a rigid back-support, such as on the ground or a floor, or in a backless chair, bench or the like. For example, spectators at athletic games often sit for extended periods of time on backless benches. Similarly, hunters or other outdoorsmen may be required to sit on various types of backless supports when hunting, eating, sitting by a fire, and the like. Pregnant women and women in their postpartum period also suffer from back pain due to increased body weight and poor posture. These individuals suffer discomfort due to a lack of support in the back area. Some even find it difficult to go about their daily activities without adequate pain relief.

Certain back-support systems have been developed to ease back discomfort. However, such back-support systems suffer several drawbacks. A major drawback of these back-support mechanisms is that they do not have a multifunctional purpose. They become useless when the user no longer has back discomfort. Another major disadvantage is that they cannot be used within a garment. Yet another drawback is that they are not discrete and can be seen on the person. And another disadvantage is that they typically cannot be used when standing or on the move. They also have the drawback of having a fixed thickness or firmness making it almost impossible to cater to the varying needs of the user.

Some other back-support mechanisms are in the form of inflatables that require time and energy to inflate. Very few back-support systems allow mobility, and these are typically in the form of braces or belts and have to be worn over the abdomen which can cause unnecessary discomfort and irritation. The existing back support mechanisms are also lacking in customizability, restricting the options to use the back support in different positions and orientations. All of the aforementioned disadvantages work together to increase the need for an improved back-support system.

Those concerned with these and other problems recognize the need for a novel and improved back-support mechanism that is capable of providing customizable back-support that is easy and comfortable to use. A further need exists for flexible back-support mechanism that provides highly portable heat and/or cold therapy. The option to use the back-support system for other purposes such as storage further increases its usability.

### SUMMARY OF THE INVENTION

A portable back-support apparatus that comprises a pad for pain relief is disclosed. In preferred embodiments, this pain-relief pad comprises one or more materials that retain their temperature for a significant period of time. The back-support apparatus has a strap attached to it, which makes it possible to attach the back-support to a chair or a seat when necessary. In further preferred embodiments the back-support apparatus comprises a rollable and/or foldable pad, hereafter referred to as flexible pad.

The flexible pad can be rolled up or folded to provide customizable support to the user's back. In the rolled state, the flexible pad provides functionality similar to a lumbar roll. The flexible pad can be attached to a garment by means of hook and loop type fasteners (commonly known by the trademark VELCRO), adhesive tape, snaps or any other fastening means. The back-support mechanism supports the back region of the wearer, while seated, standing or moving.

In further preferred embodiments, the flexible pad comprises one or more pockets. The one or more pockets may be used for storage, or for adding material that could allow modifying the firmness and thermal properties of the flexible pad. The material may be solid, liquid, semi-solid or gas.

In further preferred embodiments, the one or more pockets in the flexible pad comprise one or more closures, flaps or valves to ensure that the contents of the one or more pockets do not escape or leak.

In other embodiments, the flexible pad comprises one or more removable sections for decreasing the thickness of the back-support mechanism when it is in a rolled or folded state. The one or more removeable sections can be reattached to the flexible pad using one or more fasteners.

In further embodiments, the one or more fasteners enable the removable sections to be reattached to the flexible pad in different positions and orientations. In such embodiments, the thickness of the back-support mechanism could be customized to the comfort of the wearer even when the flexible pad is not in a rolled or folded state.

In further embodiments, the removable sections comprise one or more pockets. In such embodiments, the one or more pockets may be used for storage, or for adding material that could allow modifying the firmness and thermal properties of the flexible pad. The material may be solid, liquid, semi-solid or gas.

In still further embodiments, the one or more pockets in the removable sections comprise one or more closures, flaps or valves to ensure that the contents of the one or more pockets do not escape or leak.

### BRIEF DESCRIPTION OF THE DRAWINGS

Some embodiments of the present invention are illustrated as an example and are not limited by the figures of the accompanying drawings, in which like references may indicate similar elements and in which:

FIG. 1A represents the front view of the flexible pad 2 with pockets 3, 5 and 7 with closures 9.11 and 13. A strap 16 maybe attached to the flexible pad 2.

FIG. 1B represents the back side 6 of the flexible pad 2 with fastening means 8.

FIG. 1C depicts the flexible pad 2 when it is rolled with an attached strap 16.

FIG. 1D represents the flexible pad 2, with pockets 5 and 7 are stacked one on top of the other.

FIG. 1E illustrates the flexible pad 2 in a folded state, with strap 16.

FIG. 2A depicts an inside view of a garment 10, with an inner central portion 12 and fastening means on the lower back portion 14.

FIG. 2B represents a view of the flexible pad 2 when it is rolled up to form a back-support mechanism and fastened to the inside of a garment.

FIG. 3 illustrates a view of a chair 20 with the back-support mechanism 2 attached to it.

### DETAILED DESCRIPTION

A back-support mechanism to help people who suffer from back pain and discomfort is disclosed. This back-support mechanism helps the user to be supported on the back in multiple ways. Moreover, the back-support mechanism can be used as a pocket within or without a garment as well.

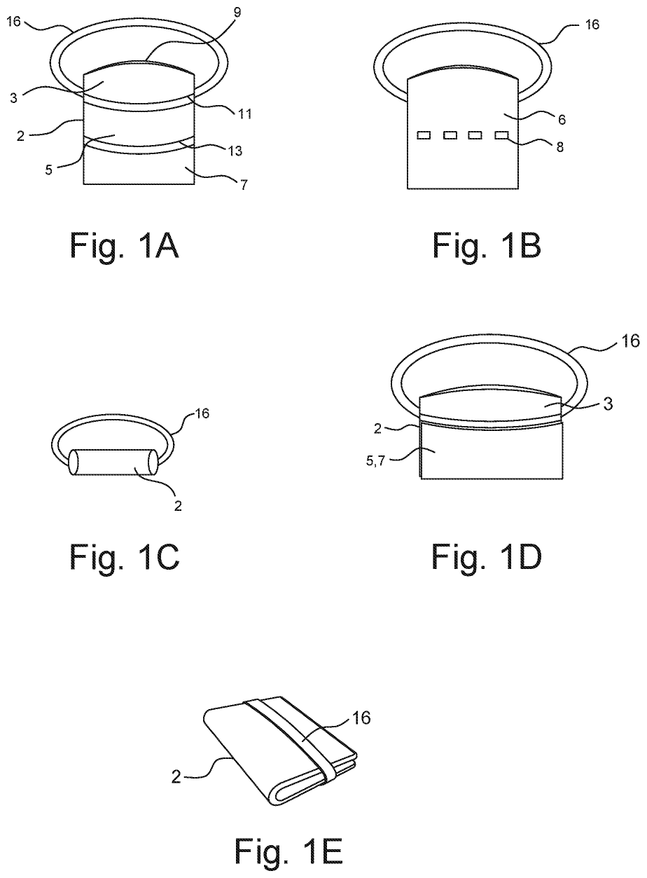

FIG. 1A. shows the front view of the back-supporting mechanism, which may be in the form of a flexible pad 2. Solid materials such as foam, semi-solid materials such as gel, liquids such as water and air could be used to provide back-support in flexible pad 2. This pad can either be rolled up or folded in different ways. This pad has at least one pocket, but shown here with three pockets 3, 5 and 7. These pockets may or may not have closures, but are shown here with closures 9, 11 and 13 respectively. Each of these pockets may be secured to each other with releasable mutually engageable end fastening means.

In further preferred embodiments, the flexible pad 2 may be capable of providing heat and/or cold therapy. In such embodiments, the flexible pad comprises material that may be capable of retaining hot or cold temperatures for a substantial period of time. An example of materials that have this thermal property is Phase Change Materials (PCM). PCMs are capable of storing and releasing large amounts of thermal energy. When the PCM's temperature is lower than the surrounding area, heat transfers from the surroundings to the material, which creates a cooling effect. When the PCM's temperature is higher than that of the surrounding area, heat transfers from the PCM to the surroundings, generating a warming effect. The flexible pad can be warmed up in a microwave or frozen in a refrigerator. The outer layer of the flexible pad maybe made of microwave and refrigerator safe materials or fabrics. For additional support, the pockets in the flexible pad 2 could be filled with additional material to adjust the firmness or thickness of the flexible pad 2. In some embodiments, the additional material may be air or water. In such embodiments, a valve prevents the leaking of the additional material. In further preferred embodiments, a strap 16 may be attached to the flexible pad 2.

FIG. 1B. depicts the back view of the flexible pad 2. One or more snaps, hook and loop type fasteners 8 or other similar fastening means may be placed on the back side of the flexible pad 2 to hold it in a rolled up state or a folded state.

FIG. 1C. depicts the flexible pad 2, with pockets 3, 5 and 7, when it is rolled up with an attached strap 16. To reduce the thickness of the roll, at least one of the pockets could be removed. FIG. 1D. depicts how the flexible pad 2 can be customized by repositioning one or more pockets from the pad to adjust the thickness of the back-support mechanism according to individual preferences. For example, pocket 7 has been removed from the original position in flexible pad 2 and placed on pocket 5 to give added thickness or firmness to the flexible pad 2 for better comfort. Pockets 3, 5 and 7 in the flexible pad 2 have releasable mutually engageable end fastening means on all four sides that allow the pockets to be easily detached from one another and easily attached to the remaining part of the flexible pad in different orientations and positions. FIG. 1E. illustrates the flexible pad 2 with a strap 16, when it is in a folded state.

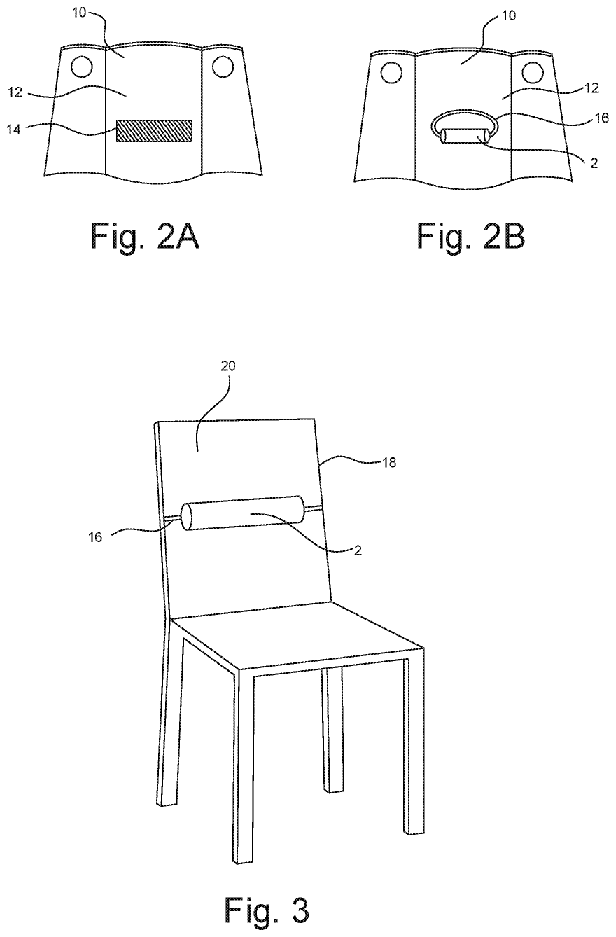

FIG. 2A. depicts the inner central portion 12 of a garment 10. In this figure, flexible pad 2 is attached to the inner portion 12 of a garment using cooperating fastener means 14, which could be in the form of one or more hook and loop fasteners, snaps and the like. The cooperating fastener means 14 could be placed in such a manner so as to customize the positioning of the flexible pad for maximum comfort. In some other embodiments, the flexible pad 2 is attachable to a garment, even if the garment does not have fastener means. In such instances, an adhesive tape may be used. FIG. 2B. depicts the inner central portion 12 of a garment 10, with the flexible pad 2 rolled up to form a back-support mechanism. The flexible pad 2 can be placed either horizontally or vertically in the rolled-up state within a garment.

In preferred embodiments, a strap 16 maybe attached to the flexible pad 2. FIG. 3. represents a view of a chair 20 with the flexible pad 2 attached to the backrest 18 of a chair 20. The strap 16 on the flexible pad 2, allows the flexible pad to be fixed to the backrest of a chair. The strap could be made of elastic material.

The current application has many advantages over existing back-support devices including at least the following: (1) The back-support mechanism can be used as a heating and/or cooling pad or rolled up to provide functionality similar to a lumbar/roll. (2) The back-support mechanism comprises at least of one pocket. (3) The back-support mechanism can be used in a garment in a concealed manner. (4) The back-support mechanism allows for customization—the wearer chooses the placement of the back-support mechanism. (5) It can be used as a standalone piece on a chair or car seat. (6) The back-support mechanism is compact enough to be incorporated into any garment; (7) The back-support mechanism is discrete and may be used without attracting attention of the public. (8) The braces or belts described in the prior art go over the abdomen of the user, which could be uncomfortable to the user. (9) The current device is re-usable. (10) Back-support devices such as lumbar rolls do not have a customizable shape.

While the above is a complete description of the preferred embodiments of the present invention, it is possible to use various alternatives, modifications and equivalents. Therefore, the scope of the present invention should be determined not with reference to the above description but should, instead, be determined with reference to the appended claims, along with their full scope of equivalents. Any feature described herein, whether preferred or not, may be combined with any other feature described herein, whether preferred or not. In the claims that follow, the indefinite article “A”, or “An” refers to a quantity of one or more of the items following the article, except where expressly stated otherwise. The appended claims are not to be interpreted as including means-plus-function limitations, unless such a limitation is explicitly recited in a given claim using the phrase “means for.”

What is claimed is:

1. A back-support system comprising:
a flexible pad;
at least one strap attached to said flexible pad; and
at least one fastener configured to removably attach one part of said flexible pad to another part of said flexible pad for holding the pad in a rolled or folded state.

2. A back-support system comprising:
a flexible pad;
at least one strap attached to said flexible pad;
at least one first fastener configured to removably attach one part of said flexible pad to another part of said flexible pad for holding the flexible pad in a rolled or folded state; and
at least one second fastener configured to removably attach the flexible pad to the inner surface of a garment.

3. The back-support system according to claim 2 wherein the at least one strap is made of elastic material.

4. The back-support system according to claim 2 wherein the flexible pad comprises a thermal management material which can be heated or cooled to a pre-determined temperature, wherein said pre-determined temperature is substantially maintained for a substantial period of time.

5. The back-support system according to claim 4 wherein the thermal management material comprises a phase change material.

6. The back-support system according to claim 2 wherein the flexible pad comprises a removable portion to reduce the surface area of the back-support.

7. The back-support system according to claim 2 wherein the flexible pad comprises at least one opening.

8. The back-support system according to claim 7 wherein the at least one opening is configured for storage.

9. The back-support system according to claim 7 wherein the at least one opening can hold additional material to adjust the firmness or thickness of the back-support.

10. The back-support system according to claim 7 wherein the at least one opening can hold at least one material capable of retaining hot or cold temperatures for a substantial period of time.

11. The back-support system according to claim 7 wherein the flexible pad further comprises at least one closure for the at least one opening.

12. The back-support system according to claim 7 wherein the flexible pad further comprises at least one valve for controlling fluid flow through the at least one opening

13. A back-support system comprising:
a pain-relief pad comprising a thermal management material which can be heated or cooled to a pre-determined temperature, wherein said pre-determined temperature is substantially maintained for a substantial period of time; and
a fastening means configured to releasably engage the pain-relief pad with the inner surface of a garment.

14. The back-support system according to claim 13 wherein the fastening means comprises a plurality of hooking elements configured to catch fibers on the inner surface of a garment.

15. The back-support system according to claim 13 wherein the fastening means comprises an adhesive tape configured to releasably engage with the inner surface of a garment.

16. The back-support system according to claim 13 wherein the fastening means comprises mutually engageable first and second fasteners, the first fastener being affixable to the pain-relief pad and the second fastener being affixable to the inner surface of a garment.

17. The back-support system according to claim 16 wherein the fastening means comprises hook and loop fasteners.

18. The back-support system according to claim 16 wherein the fastening means comprises magnetic coupling.

19. The back-support system according to claim 13 wherein the pain-relief pad further comprises a strap.

20. The back-support system according to claim 13 wherein the thermal management material comprises a phase change material.

21. The back-support system according to claim 13 wherein the pain-relief pad comprises at least one fastener configured to removably attach one part of said pain-relief pad to another part of said pain-relief pad for holding the pain-relief pad in a rolled or folded state.
